(19) United States
(12) Patent Application Publication
(10) Pub. No.: US 2023/0250951 A1
(43) Pub. Date: Aug. 10, 2023

(54) AIR TREATMENT APPLIANCE CONFIGURED AS A SUBSTITUTE FOR A LIGHT BULB OR FOR A HANGING LAMP

(71) Applicant: Airfree Produtos Electronicos S.A., Lisbon (PT)

(72) Inventor: Carlos Jose Duarte Matias, Lisbon (PT)

(21) Appl. No.: 17/697,359

(22) Filed: Mar. 17, 2022

Related U.S. Application Data
(60) Provisional application No. 63/307,254, filed on Feb. 7, 2022.

(51) Int. Cl. F21V 33/00; F21K 9/232; A61L 9/16

(52) U.S. Cl. CPC F21V 33/0096; F21K 9/232; A61L 9/16; F21Y 2115/10

(57) ABSTRACT

An air treatment appliance is shown situated in a container, wherein the air treatment appliance includes an electrical power absorber; and includes an electrical interface configured to interface the container with an electrical lighting fixture normally configured for lighting a room with a lamp, the interface connected directly or indirectly to the power absorber for providing electrical current to the electrical power absorber for performing an air treatment action that treats air in or around the appliance or treats air in and around the appliance, wherein the electrical interface is a standarized interface that interfaces with a mating interface of the electrical lighting fixture.

### BACKGROUND OF THE INVENTION

One of the major issues in most homes and confined spaces in public structures is the difficulty in placement of air treatment devices such as air purifiers because they occupy floor space and/or space on top of furniture that could otherwise be left empty or employed for another purpose.

In the context of the present invention, air treatment appliances are for use in confined spaces. Dfor instance, an air purifier appliance treats a flow of air by heating it to a sufficiently high temperature to eliminate hamful airborne particles, bacteria, or the like, to thereby purify the air in the confined space.

### SUMMARY OF INVENTION

An object of the present invention is to free up space such as floor and furniture-top space where usually air treatment apppliances are placed.

The present application answers such a need, by simply allowing such air treatment appliances, by means of an electrical interface associated therewith, to be connected to an electrical energy source via a fixture useable for supporting and energizing another device. An example is an air treatment appliance configured with an electrical interface that permits it to utilize an electrical interface of a light fixture, as a substitute or replacement for a light bulb or lamp; that is, with an electrical interface that matches the interface used in a fixture traditionally intended for use in supporting and energizing a light bulb or lamp. The electrical interface of the appliance in one such case is a metallic lamp or light bulb base. Such bases are sometimes referred to as “caps,” and include dozens of types, the most common being Edison screw bases with a cylindrical shape and a male right hand thread on its metallic surface. The medium or standard base E26 (for 120-volt North America) and E27 (for 220-240 volt Europe) are usually interchangeable with the same or almost the same thread outside diameter and thread pitch. The standard base is screwed into a matching female threaded socket (lamp holder) that is typically connected to a room or appliance switch with which the socket is energized by electrial power. For such AC powered lamps, the thread is usually connected to neutral and the contact at the bottom to the “hot” phase. There are many Edison base type variations such as miniature candelabra, medium screw, mogul screw, mogul exclusionary, etc., which are not always interchangeable internationally. There are other types as well such as single or double-contact bayonet, FA8, G4, etc.

The mating electrical interface with which the appliance's electrical interface mates may be installed in fixtures that take different forms. Examples are an existing ceiling fixture or hanging lamp or a table or wall lamp. In other words, the typical lamp or light bulb is not installed in the fixture for the purpose of lighting but an air treatment appliance is substituted and facilitated by its electrical interface configured so as to function in the lamp fixture as a replacement light bulb, lamp, or equivalent.

An example of an air treatment appliance combined with an electrical interface according to the invention is an air sterilization appliance with an Edison screw base built into one end. Such an air sterilization appliance may be in the form of a heat-resistant body equipped internallly with mini-ducts configured to contain intense heat which effectively kills micro-organisms such as viruses, mold, and bacteria, without the release of large quantities of heat into the environment. Such may form part of an air sterilization system including several air sterilization appliances for use indoors, for instance in a room, and is aimed to kill bacteria, to avoid the development of mold and mildew and the like, without occupying any space on top of furniture, on the floor or mounted on walls.

Such an air sterilization appliance may consist of a sterilization ceramic core consisting of multiple mini-ducts of small diameter preferably heated by at least one electrically resistant wire that passes in the mini-ducts. The electrically resistant wire is connected to a power supply via a switch in a room or in the fixture in which the air treatment appliance is installed. When electrical current runs through the wire, the resistance of the wire generates heat, which is radiated into the air surrounding the wire inside the mini-ducts. The electrical energy delivered from the power source via the resistant wire is designed to provide heat inside the mini-ducts in excess of 180° Celsius. The heat inside the mini-ducts, when the resistant wire is connected to the exterior power source generates a heated air stream that flows upwardly by means of heating the air contained therein when the ceramic pipe is in its preferably vertical position. When the heated air exits the mini-ducts a negative force is created at the bottom of the mini-ducts dragging exterior air into the ceramic mini-ducts and therefore creating a continuous air circulation through the mini-ducts. Airborne micro-organisms are exterminated by heat when passing inside the heated mini-ducts. The continuous airflow generated by the air convection as above described assures 99.99% air sterilization in a quiet and efficient way and with low power consumption. The tubes are preferably made of a good quality ceramic or material that can stand heat well above 200° C. and allow the mini-ducts to be as close as possible to allow heat interchange between them. The ceramic core is installed into an exterior casing equipped with easy air access at the bottom and at the top. An optional heat exchanger can be used at some distance over the air exhausting top of the ceramic mini-ducts and a casing top made of a material resistant to heat, with at least one air outlet that will preferably boost air speed out. The casing is preferably constructed to incorporate the ceramic core and to preferably be structured to be installed in a fixture installed in or hanging from the ceiling or installed in a table, wall, or floor lighting fixture.

### BRIEF DESCRIPTION OF THE DRAWINGS

FIG. 1A shows an apparatus that includes an air treatment appliance configured as a lamp, according to the present invention, connected to an electrical interface.

FIG. 1B shows an embodiment of the apparatus of FIG. 1A in the form of an air sterilization appliance with a cutaway side view showing internal details.

FIG. 1C shows an embodiment similar to that of FIG. 1B but with more and different details, also shown with a cutaway, but with an upward perspective view.

FIG. 2A shows an air treatment appliance configured as an air sterilization assembly having a container that is affixed or inserted into the ceiling of an elevator or other closed room setup where sterilized air exhausts through air outlet to a chamber like an elevator shaft or both to the elevator and the elevator shaft or in general to a space above the ceiling.

FIG. 2B shows a cross section of the air sterilization assembly attached to an elevator top wall or ceiling 50.

FIG. 2C shows a cross section of the air sterilization assembly of FIG. 2A with one possible embodiment of a bottom lid allowing a fan to be attachd to the botom lid along with multiple filters such as for particles, VOC's and other airborne contaminants.

In FIG. 3, a container is coupled to a ventilation duct, protected by a deflector plate to avoid the air flow interfering with the upward flow of sterilized air coming from the air sterilization assembly top so that it is assured that the sterilized air is pumped back into the room though a ventilation system of which the duct is a part.

In FIG. 4 a container of a treatment appliance in the form of a hanging lamp (not shown) is connected to a socket in the ceiling.

FIG. 5 shows an air treatment appliance configued as a “lamp” with a light bulb electrical interface screwed into an electrical lamp socket at the top end of a column of a table lighting fixture with an electrical wire inside the column.

FIG. 6 shows an air treatment appliance with a threaded cap 600 at the bottom of a container 602 shown in cutaway to show the central core within.

### DETAILED DESCRIPTION OF THE PREFERRED EMBODIMENTS and FIGURES

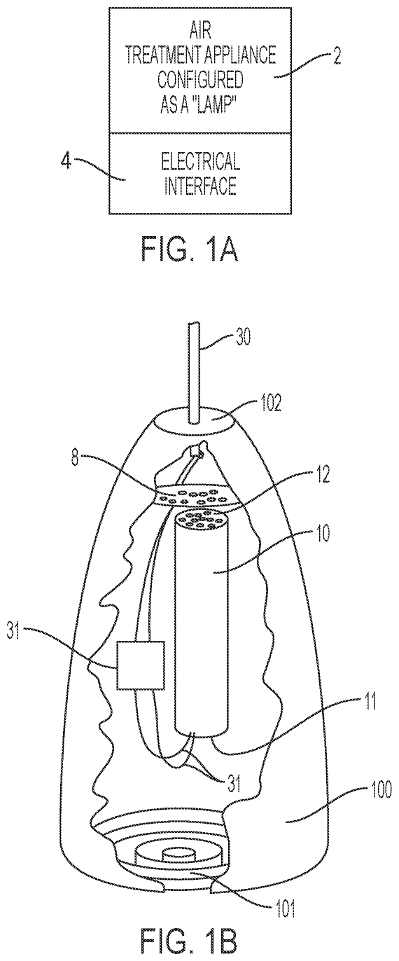

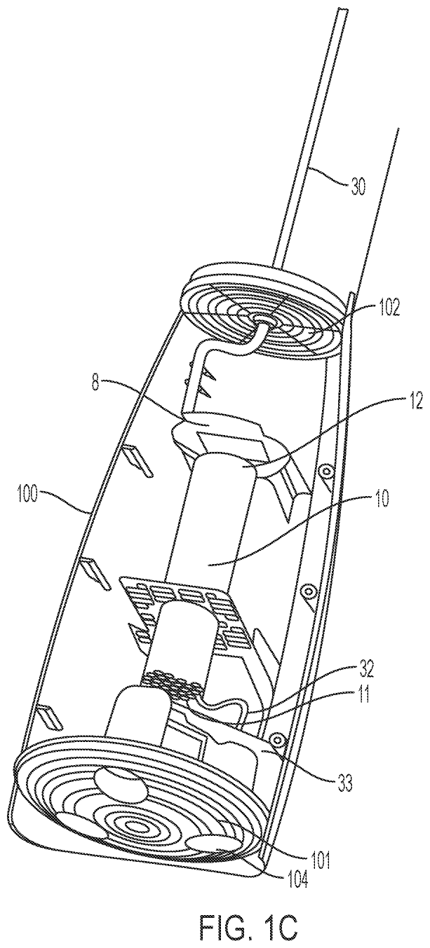

FIG. 1A shows an apparatus that includes an air treatment appliance 2 configured as a lamp, according to the present invention, connected to an electrical interface 4. The apparatus may include a container, an air treatment appliance situated in the container, wherein the air treatment appliance includes an electrical power absorber; and an electrical interface configured to interface the container with an electrical lighting fixture configured for lighting a room with a lamp, the interface connected directly or indirectly to the power absorber for providing electrical current to the electrical power absorber for performing an air treatment action that treats air in or around the appliance or that treats air in and around the appliance.

The electrical interface may be a standardized interface that interfaces with a mating interface of the electrical lighting fixture. The air treatment appliance need not necessarily be contained in a container. In other words, the container may be omitted in some embodiments.

An example is an air treatment appliance configured with an electrical interface that permits it to utilize an electrical interface of a light fixture, as a substitute or replacement for a light bulb or lamp; that is, with an electrical interface that matches the interface used in a fixture traditionally intended for use in supporting and energizing a light bulb or lamp. The electrical interface of the appliance in one such case is a metallic lamp or light bulb base. Such bases are sometimes referred to as “caps,” and include dozens of types, the most common being Edison screw bases with a cylindrical shape and a male right hand thread on its metallic surface. The medium or standard base E26 (for 120-volt North America) and E27 (for 220-240 volt Europe) are usually interchangeable with the same or almost the same thread outside diameter and thread pitch. The standard base is screwed into a matching female threaded socket (lamp holder) that is typically connected to a room or appliance switch with which the socket is energized by electrial power. For such AC powered lamps, the thread is usually connected to neutral and the contact at the bottom to the “hot” phase. There are many Edison base type variations such as miniature candelabra, medium screw, mogul screw, mogul exclusionary, etc., which are not always interchangeable internationally. There are other types as well such as single or double-contact bayonet, FA8, G4, etc.

The mating electrical interface with which the appliance's electrical interface mates may be installed in fixtures that take different forms. As shown in more detail below, examples are an electrical interface to an existing ceiling fixture or hanging lamp or a table or wall lamp. In other words, the typical lamp or light bulb is not installed in the fixture for the purpose or sole purpose of lighting but an air treatment appliance is substituted in whole or in part, facilitated by its electrical interface configured so as to function in the lamp fixture as a replacement light bulb, lamp, or equivalent.

The electrical interface 4 shown in FIG. 1A may include electronics to aid in control of the appliance 2. Such may include one or more sensors, sampling devices, analog-to-digital (A/D) and/or digital-to-analog (D/A) conversion devices, a control chip such as a microcontroller or the like, or even a full-fledged microprocessor, along with appliance actuator control devices and any other device or devices needed to carry out the operation of the appliance.

As described in more detail below, an example of an air treatment appliance combined with an electrical interface according to the invention is an air sterilization device appliance with an Edison screw base built into one end. Such an air sterilization appliance may include a container that contains an air sterilzation body, e.g., in the form of a heat-resistant cylindrical body equipped internallly with a plurality of mini-ducts aligned in parallel with the axis of the cylinder and configured with a heated wire to supply intense heat within the mini-ducts which effectively kills micro-organisms such as viruses, mold, and bacteria, without the release of large quantities of heat into the environment. Such may form a part of an air sterilization system including several air sterilization appliances for use indoors, for instance in a room, and is aimed to kill bacteria, to avoid the development of mold and mildew and the like, without occupying any space on top of furniture, on the floor or mounted on walls.

In more detail, such an air steriliszation device appliance may include an air sterilization ceramic core that has multiple mini-ducts of small diameter preferably heated by at least one electrically resistant wire that is threaded and passes through the mini-ducts. The electrically resistant wire is connected to a power supply via a switch in a room or in the fixture in which the air treatment appliance is installed. When electrical current runs through the wire, the resistance of the wire generates heat, which is radiated into the air surrounding the wire inside each of the mini-ducts. The electrical energy delivered from the power supply source to the air treatment appliance via the electrical interface to the resistant wire is designed to provide a heat buildup inside the mini-ducts in excess of 180° Celsius. The heat inside the mini-ducts, when the resistant wire is plugged into and connected to the exterior power source generates an upward flow or stream of air by means of heating the air there contained when the ceramic pipe is in its preferably vertical position. When the heated air exits the mini-ducts a negative force is created at the bottom of the mini-ducts dragging exterior air into the ceramic mini-ducts and therefore creating a continuous air circulation through the mini-ducts. Airborne micro-organisms are exterminated by the heat when passing inside the heated mini-ducts. The continuous airflow generated by the air convection as above described assures 99.99% air sterilization in a quiet and efficient way and with low power consumption. The tubes are preferably made of a good quality ceramic or material that can stand heat well above 200° C. and allow the mini-ducts to be as close as possible to allow heat interchange between them. The ceramic core is installed into an exterior container or casing that may be made to resemble a lamp and equipped with easy air access at the bottom and exit at the top. An optional heat exchanger can be used at some distance over the air exhausting top of the ceramic mini-ducts and a casing top made of a material resistant to heat, with at least one air outlet that will preferably boost air speed out the top. The casing is preferably constructed to incorporate the ceramic core and to preferably be structured to resemble a lighting fixture installed in or hanging from the ceiling or installed in a table lamp fixture, wall lamp fixture, or floor lamp fixture or any similar lighting fixture or structure.

FIG. 1B shows an embodiment of the apparatus of FIG. 1A in the form of an air treatment appliance. The air treatment appliance is situated in a container 100 that is outwardly configured with the appearance of a hanging lamp. In the particular embodiment of FIG. 1B, the air treatment appliance includes an air sterilizer assembly. As shown, the apparatus is configured to hang from a ceiling or any other stucture by means of a power cord 30 that provides part of an electrical interface. Thus a typical lamp or light bulb is not installed at the end of power cord 30 as would be usual for a hanging lamp fixture, i.e., a fixture for the purpose of lighting, but rather an air treatment appliance is substituted. As shown within by the cutaway perspective side view, the substitution is effected by using the power cord 30 as an interface to a substituted air treatment appliance. The electrical interface may also include a device 31 to assist in the air treatment. Such may invclude a printed circuit board with various components such as a rectifier, a controller, etc. Thus the combined electrical interface 30, 31 acts with the air treatment appliance configured as a lamp-like device to effect the air treatment of sterilization. The air treatment by means of the air treatment appliance configured as a lamp is thus facilitated by its electrical interface 30, 31 and the combined appliance/interface function in the container as a replacement light bulb, lamp, or equivalent. The container of FIG. 1B has contaminated air entering by air convection at an inlet 101 which may include louvered, concentric circular openings. The air in sequence enters at a bottom end 11 of a sterilizing ceramic element 10 of the type shown and described in Applicant's prior U.S. Patents, for instance, U.S. Pat. No. 5,874,050. The hot sterilized air exhausts at the sterilizing element top end 12. An optional heat exchanger 8 can be added to cool the sterilized air exhausting from the top end 12 of the ceramic element 10, before passing through an air outlet 102 at the top into the room.

FIG. 1C shows an embodiment similar to that of FIG. 1B but with differing details, also with a cutaway, but with an upward perspective view in order to show lamps 109 (which may be LEDs) arranged on the bottom. Like FIG. 1B, the air treatment appliance is situated in a container 100 that is outwardly configured with the appearance of a hanging lamp. In the embodiment of FIG. 1C, the air treatment appliance also includes an air sterilizer assembly. As shown, the apparatus is configured to hang from a ceiling or any other stucture by means of a power cord 30 that provides part of an electrical interface. Thus a typical lamp or light bulb is not installed by itself at the end of power cord 30 as would be usual for a hanging lamp fixture, i.e., a fixture for the purpose of lighting alone, but rather an air treatment appliance is substituted as in FIG. 1B but also including one or more lamps 109 arranged at the bottom. As shown within by the cutaway perspective bottom view of FIG. 1C, the transformation is effected by using the power cord 30 as an interface to a substituted air treatment appliance with one or more lamps added as an option. The electrical interface may also include a device 33 to assist in the air treatment. Such may include a printed circuit board 33 with various components such as a rectifier, a controller, etc. Electrical current is provided on a wire 32. Thus the combined electrical interface 30, 32, 33 of FIG. 1C altogether function along with the air treatment appliance configured as a lamp-like device to effect the air treatment of sterilization. The air treatment by means of the air treatment appliance configured as a lamp is thus facilitated by its electrical interface 30, 32, 33 and the combined appliance/interface function in the container as a replacement light bulb, lamp, or equivalent. As mentioned, it may also include one or more lamps such as the three LED lamps 109 arranged symmetrically around the base of the container. These lamps 109 may be controlled separately from the air treatment appliance within the container 100. This could be via a switch (not shown) on the container. Or a separate pair of wires could be run in the pipe 30 that is controlled remotely to deliver power to the lamps independently from the power delivered on another pair of wires connected to the air treatment appliance. The lamps 109 may be permanent or replaceable. As with FIG. 1B, the container of FIG. 1C has contaminated air entering by air convection at an inlet 101 which may include louvered, concentric circular openings. The air in sequence enters at a bottom end 11 of the sterilizing ceramic element 10. The hot sterilized air exhausts at the sterilizing element top end 12. An optional heat exchanger 8 can be added to cool the sterilized air exhausting from the top end 12 of the ceramic element 10, before passing through an air outlet 102 at the top and thence into the room.

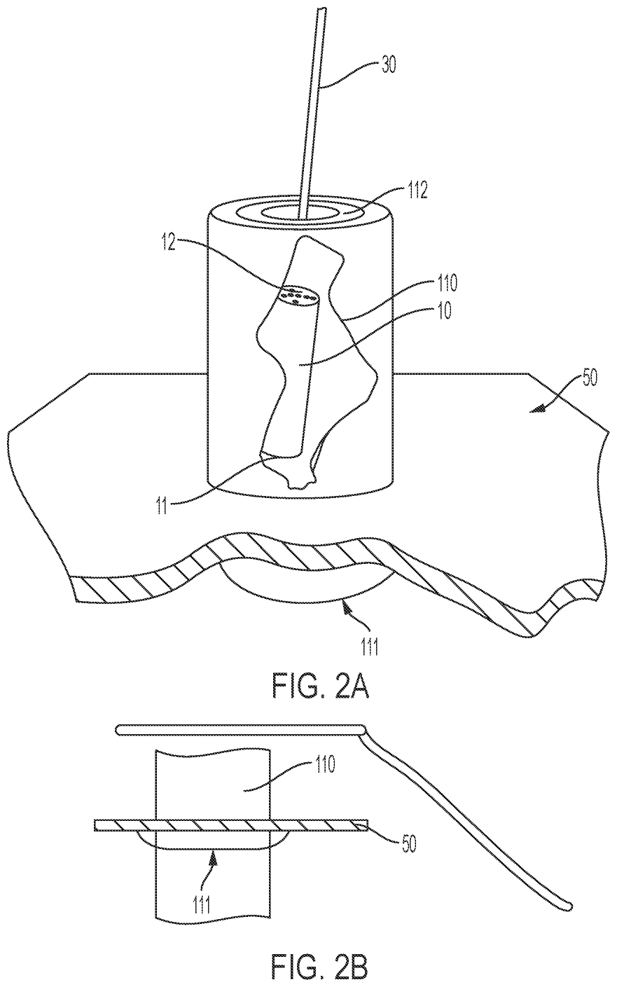

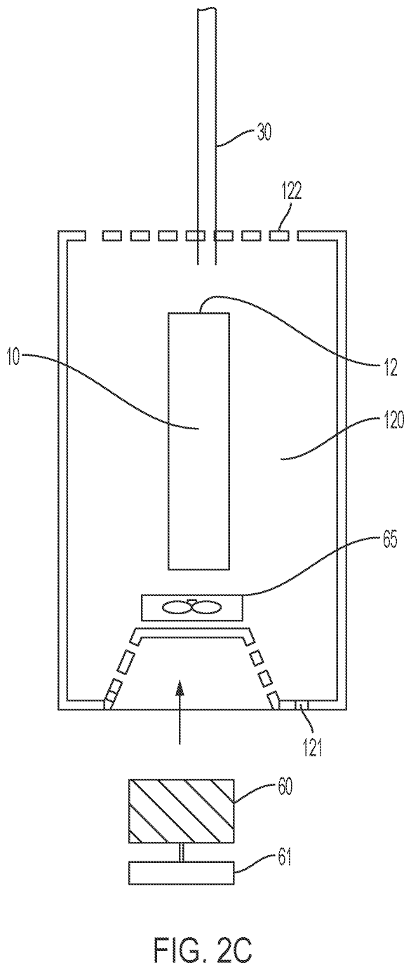

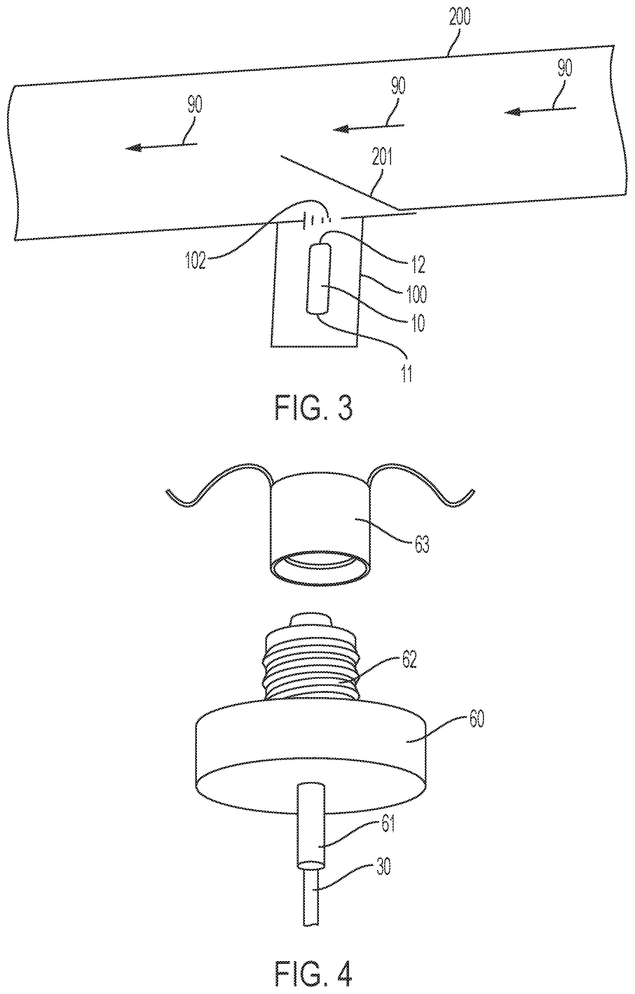

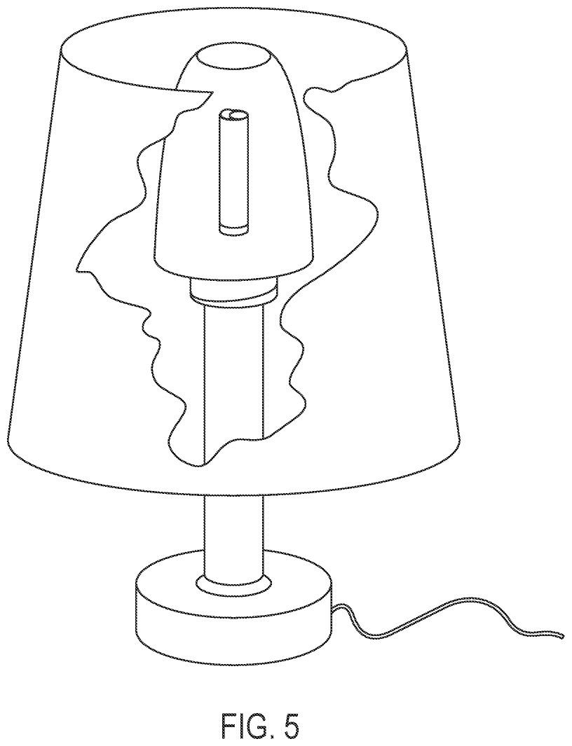

As shown in the cutaway view of FIG. 2A, an air treatment appliance is configured as an air sterilization assembly having a container 110 that is affixed or inserted into the ceiling of an elevator 50 or other similar closed-room setup where sterilized air exhausts through air outlet 112 to a chamber like an elevator shaft or both to the elevator and the elevator shaft.

FIG. 2B shows a cross section of the air sterilization assembly attached to an elevator top wall or ceiling 50. FIG. 2C shows a cross section of the air sterilization assembly with one possible embodiment of a bottom lid 121 allowing a fan 65 to be attached to said botom lid along with multiple filters 60 and 61 such as for particles, VOC's and other airborne contaminants. An air outlet 122 is provided at the top.

Although not shown in FIGS. 2A, 2B, and 2C, it should be understood that an electrical interface 4 is provided for the illustrated embodiment such as shown in FIG. 1A or FIG. 1B in combination with the air treatment appliance configured as a lamp.

In the embodiment of FIG. 3, a container 100 is coupled to a ventilation duct 200, protected by a deflector plate 201 to avoid the air flow 90 interfering with the upward flow of sterilized air coming from the air sterilization assembly top 102 so that it is assured that the sterilized air is pumped back into the room though a ventilation system of which the duct 200 is a part. Although not shown in FIG. 3, it should be understood that an electrical interface 4 is provided such as shown in FIG. 1A, FIG. 1B, or FIG. 1C in combination with the air treatment appliance configured as a lamp.

In FIG. 4 a container of an air treatment appliance in the form of a hanging lamp (not shown) is connected to an electrical socket 63 in the ceiling. The electrical interface in this case is configured as a light bulb type of connector 62 coupled to an electrical source via the socket 63. Thus, the top part 62 of the electrical interface 60, 61 is simply twisted into an existing lamp receptacle or socket 63, with power cord 30 passing through pipe 61 to the air sterilization appliance in a container configured to resemble a hanging lamp, not seen in the figure. A disc part 60 of the electrical interface 4 may include electronics such as a rectifier, a microcontroller, and the like for receiving sensed feedback signals that sense temperature and using that feedback in regulating the operation of the air treatment apparatus at the end of the pipe 61 and cord 30.

In each of these embodiments the electrical connections to the air sterilizing units are not shown in detail. These are conventional connections, such as those of the type already shown in Applicant's prior U.S. Patents.

With the use of the new air sterilization system as shown in the various figures, air circulation through the air sterilization assemblies is created by air convection through the use of air sterilizers similar to those used in room air sterilizer devices invented by Applicant in previous U.S. Patents but also with said air flow can be enhanced by coupling fans.

The air technology utilized offers excellent results with exceptional reduction and destruction of viruses, bacteria, molds and allergen and improvement in indoor air quality as proven by international laboratories liks SGS, CMA Testing, Intertek—INETI—Laboratory of Microbiology in Lisbon, Segamb, Pedamb, NIOSH, Universidad Complutense of Madrid, TMC—Technical Micronics Corporation in the United States of America and other tests. These tests showed that the sterilization assembly system operated in a highly improved fashion when compared to systems relying on filters, and chemical agents. No air sterilizing or air purifying systems are available up to today for refrigerators and cooling chambers.

Other preferred embodiments of the invention can work with multiple air treatment appliances configured as air sterilization appliances in the same confined space. The system can either operate with the natural air convection occurrence caused by air heating at the mini-ducts of the air sterilizing assembly ceramic element or by a dedicated air flow system associated with the air sterilization assembly. The dedicated air flow system may include a fan to blow air into the air sterilization assembly input port or a fan to pull air from the output port.

To minimize the effect of the heat utilized in the air sterilization assembly, the system may include an additional cooling chamber inside or outside of the air sterilization assembly which cools the flow of air coming out of the ceramic core exhaust outlet prior to re-entry into the enclosed air volume such as a chamber, room or elevator.

As suggested above, in addition to being utilized in rooms, chambers or elevators, multiple air sterilization appliances can be utilized in enclosed volumes and also in connection with central air conditioning or heating air ducts. In these cases, the size, number and location of the air sterilization appliances incorporated into the system are adaptable based upon the needs of the system, including its volume, airflow, temperature, humidity and other physical characteristics. Accordingly, the air sterilization appliance can be placed either fully inside the room, chamber, compartment, elevator or inside or outside its top walls and or a partial or total mixture of said options with direct inlet and outlet connections to the room, chamber, compartment, elevator or into an elevator pit for instance where its sterilized air enter the elevator cabin while the air sterilization assembly is pulling the contaminated air from, sterilizing it and deliver it totally or partially to the elevator pit or cabin.

FIG. 5 shows an air treatment appliance configued as a “lamp” with a light bulb electrical interface screwed into an electrical lamp socket at the top end of a column of a table lighting fixture with an electrical wire inside the column. The socket, column, or base of the fixture may have an associated switch that is switchable on or off to provide electrical current.

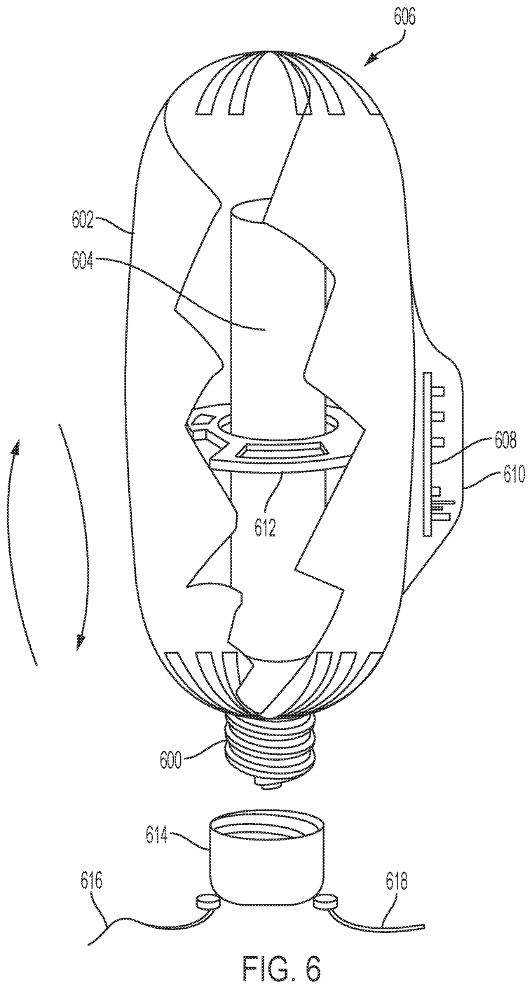

The air treatment appliance of FIG. 5 may take the form shown in FIG. 6 having a threaded cap 600 at the bottom of a container 602 shown in cutaway showing the central core 604 within. An exit 606 is provided at the top for escape of air treated by heat in the central cylindrical core 604 within the container 602. As part of the electrical interface 4, a printed circuit board 608 may be provided as shown residing in a protuberance 610 on a side of the container 602 and supplied with power through the cap 600 to control aspects of the operation such as the internal temperatures in the mini-ducts in the central core 604 where heat is generated by heating wires strung in the mini-ducts in the central core 604. A central core of this type is shown in the aforementioned U.S. Pat. No. 5,874,050. The core 604 may be held in place within the container 602 by a surrounding disc-like support 612 that may have openings as shown or not. The combined air treatment appliance and its electrical interface may be screwed into a socket 614 that is supplied with electrical current via wires connected to the electrical mains by a switch. The electrical interface may include a fan and the load absorber.

Referring back to FIG. 1A, the combined electrical interface and air treatment appliance 2 (configured as a lamp i.e. to resemble a lamp) can be embodied as various different air treatment appliances.

It should be understood that the following claims are intended to cover all of the generic and specific features of the invention herein described and that all statements of the scope of the invention which, as a matter of language, might be said to fall therebetween.

1. Apparatus, comprising
a container;
an air treatment appliance situated in the container, wherein the air treatment appliance includes an electrical power absorber; and
an electrical interface configured to interface the container with an electrical lighting fixture configured for lighting a room with a lamp, the interface connected directly or indirectly to the power absorber for providing electrical current to the electrical power absorber for performing an air treatment action that treats air in or around the appliance or treats air in and around the appliance, wherein the electrical interface is a standarized interface that interfaces with a mating interface of the electrical lighting fixture.

2. The apparatus of claim 1, wherein the air treatment appliance comprises an air sterilization appliance configured for installation in a lighting fixture instead of a lamp.

3. The apparatus of claim 2, wherein the air sterilization appliance is configured for hanging from a ceiling light fixture.

4. The apparatus of claim 1, wherein the air treatment appliance is configured for installation in a table lamp fixture or a floor lamp fixture.

5. The apparatus of claim 1, wherein the air sterilization appliance is attached to a ceiling or top wall of a contained space and sterilized air is either returned to the contained space.

6. The apparatus of claim 1, wherein the air treatment appliance includes a fan.

7. The apparatus of claim 1, wherein the electrical interface incorporates a screwing device that will attach to an standard lamp receptacle for providing electrical current.

8. The apparatusof claim 1, wherein one or more filters are included in the apparatus.

9. The apparatus of claim 1, wherein an LED or other lighting device is included in the apparatus to illuminate the room, chamber, or a table.
